(19) United States
(12) Patent Application Publication
(10) Pub. No.: US 2024/0082447 A1
(43) Pub. Date: Mar. 14, 2024

(54) Devices and Methods for Providing Nitric Oxide

(71) Applicant: Sterile State LLC, Farmington Hills, MI (US)

(72) Inventors: Megan Cecelia Frost, Trenary, MI (US); Jeffrey Millin, Trenary, MI (US)

(73) Assignee: Sterile State LLC, Farmington Hills, MI (US)

(21) Appl. No.: 18/272,533

(22) PCT Filed: Jan. 26, 2022
(86) PCT No.: PCT/US22/13887

§ 371 Date: Jul. 14, 2023

Related U.S. Application Data
(60) Provisional application No. 63/156,917, filed on Mar. 4, 2021.
(60) Provisional application No. 63/141,676, filed on Jan. 26, 2021.
(60) Provisional application No. 63/141,711, filed on Jan. 26, 2021.

(51) Int. Cl. A61L 9/015; A61L 9/18

(52) U.S. Cl. CPC A61L 9/015; A61L 9/18; A61L 2202/122

(57) ABSTRACT

A device for providing nitric oxide to a space for sanitizing or sterilizing an article is provided. The device includes a support having a surface. The support is adapted to be disposed in the space and in proximity of the article or the surface defines the space and the space is adapted to contain the article. The device further includes a nitric oxide source overlying the surface and adapted to provide nitric oxide (A) at a predetermined rate, (B) for a predetermined amount of time, (C) a predetermined dose, or any combination of (A), (B), and (C). A method for sanitizing or sterilizing an article in a space is provided. The method includes locating the article within the space. The method further includes providing nitric oxide to the space from a nitric oxide source at a predetermined rate. The method further includes exposing the article to the nitric oxide for a predetermined amount of time.

### CROSS-REFERENCE TO RELATED APPLICATIONS

This application is an International Application which claims priority to Provisional Patent Application No. 63/141,676, filed on Jan. 26, 2021, Provisional Patent Application No. 63/141,711, filed on Jan. 26, 2021, and Provisional Patent Application No. 63/156,917, filed on Mar. 4, 2021, the entire contents of which are incorporated by reference in their entirety.

### TECHNICAL FIELD

The present disclosure generally relates to devices for providing nitric oxide to a space for sanitizing or sterilizing an article. The present disclosure also relates to methods for sanitizing or sterilizing an article in a space.

### BACKGROUND

A variety of products and articles, including, for example, medical instruments, devices, and equipment, must be sterilized prior to use to prevent bio-contamination of a wound site, a sample, an organism, or the like. A number of sterilization processes are used which involve contacting the product or article with a sterilant. Examples of such sterilants include steam, ethylene oxide, hydrogen peroxide, dry heat, and the like.

Ethylene oxide, for example, is well known to generate carcinogenic species that pose significant risk for users of this sterilization equipment and the extended periods of time required to aerate these devices is extremely expensive and cumbersome.

Accordingly, it is desirable to provide a gas sterilization technique that will effectively sterilize medical devices and other articles, and replace the wide spread use of ethylene oxide sterilization. Furthermore, other desirable features and characteristics will become apparent from the subsequent summary and detailed description and the appended claims, taken in conjunction with the foregoing technical field and background.

### BRIEF SUMMARY

One non-limiting embodiment of a device for providing nitric oxide to a space for sanitizing or sterilizing an article is provided. The device includes a support having a surface. The support is adapted to be disposed in the space and in proximity of the article. The device further includes a nitric oxide source overlying the surface and adapted to provide nitric oxide (A) at a predetermined rate, (B) for a predetermined amount of time, (C) at a predetermined dose, or any combination of (A), (B), and (C).

Another non-limiting embodiment of a device for providing nitric oxide to a space for sanitizing or sterilizing an article is provided. The device includes a support having a surface. The surface defines the space and the space is adapted to contain the article. The device further includes a nitric oxide source overlying the surface and adapted to provide nitric oxide (A) at a predetermined rate; (B) for a predetermined amount of time, (C) at a predetermined dose, or any combination of (A), (B), and (C).

A non-limiting embodiment of a method for sanitizing or sterilizing an article in a space is provided. The method includes locating the article within the space. The method further includes providing nitric oxide to the space from a nitric oxide source at a predetermined rate. The method further includes exposing the article to the nitric oxide for a predetermined amount of time.

In various exemplary embodiments, the devices and methods of this disclosure provide a safe, efficient, environmentally friendly gas sanitation and sterilization of a wide range of articles such as medical devices, medical equipment, endoscopes, cell phones, mask coverings, keys, name badges, credit cards, mouth guards, baby pacifiers and teething rings, pens and pencils, sports gloves, shoes, etc. The devices and methods of this disclosure use gas phase nitric oxide to sterilize or sanitize microorganisms and infectious such as bacteria, viruses, yeast and fungi on objects under ambient temperature, pressure and humidity by appropriately enclosing the article to be sterilized or sanitized and exposing the article to nitric oxide gas.

The method includes the step of placing the article in a confining container that does not need to be sealed or put under vacuum to remove oxygen or moisture. Importantly, this gas phase sterilization can be performed under ambient conditions that does not require special environmental controls such as removal of oxygen, reduced pressure, rigorously controlled humidity or temperature. The confining containers promotes the local concentration of nitric oxide to accumulate to a predetermined level to above a minimal sanitation or sterilization for a predetermined period of time. Appropriate confining containers include plastic boxes such as those used to store personal electronics, soft plastic bags similar to autoclave bags or ethylene oxide sterilization pouches, or custom storage containers for medical devise such as endoscopes. Containers can be hermetically sealed and evacuated and held under strict humidity control and temperature regulation, but this regulation is not required for effectiveness of sanitization or sterilization of the article.

Within this confining container, a nitric oxide source is present. There is a wide variety of possible nitric oxide sources that can be used, depending on the design constraints of the desired application of the system. Nitric oxide sources can include, but are not limited to, the use of SNAP-PDMS and other nitric oxide donating polymers that use different nitric oxide moieties and different polymer base materials. The nitric oxide donors can be covalently linked to the polymer or blended into the polymer. Discrete nitric oxide donors can also be used in solid, liquid or gel forms. Examples of this include SNAP, nitrite, S-nitrosocysteine, S-nitrosoglutathione, diazeniumdiolate compounds, enzymatic generation of NO from arginine, or organitrites, biological sources such a macrophage generation, etc.

Other examples of nitric oxide sources include gas phase delivery from polymers, acidified nitrite or nitrate, nitric oxide donating molecules such as diazeniumdiolates, nitrosothiols, nitrosyl compounds, or other methods of NO generation such as enzymatic production of nitric oxide, chemical production of nitric oxide from ascorbic acid or metal catalysis, electrochemical production of nitric oxide, photolytic cleavage of bonds to release nitric oxide, direct delivery of nitric oxide gas, etc.

The trigger for initiating nitric oxide release can be thermal, light, pH, water, metal ion mediated, electrochemically initiated, ascorbic acid initiated, vibrational, ultrasound, stirring, mechanical agitation or ultrasonic, etc. The devices and methods of the disclosure provide safe and efficient use of nitric oxide for through controlled timing of exposure of the article to the nitric oxide and concentration of the nitric acid.

For example, these devices and methods are effective for nitric oxide gas phase sterilization on Staph. Epi, Staph. Aurous, MRSA, and E. Coli inoculated on paper, polymers, and stainless-steel. Generation of nitric oxide to an effective level may be based on the thermal and photolytic cleavage of SNAP-PDMS, nitric oxide generation from nitrite in the presence of cysteine in both solution and powder forms, nitric oxide generation from nitrite in the presence of glutathione in both solution and powder forms, nitric oxide generation from SNAP and nitrite with copper ions, and zinc ions, nitric oxide generation from SNAP and nitrite with copper ions, and zinc ions, thermal and photolytic cleave of SNAP in acidified and neutral solution, nitric oxide generation from SNAP in basic solutions, direct delivery of compressed nitric oxide gas, etc.

### BRIEF DESCRIPTION OF THE DRAWING(S)

FIG. 1 is a perspective view illustrating a non-limiting embodiment of a device for providing nitric oxide for sanitizing or sterilizing an article in a space.

FIG. 2 is a perspective view illustrating another non-limiting embodiment of a device for providing nitric oxide for sanitizing or sterilizing an article in a space.

FIG. 3 is a perspective view illustrating another non-limiting embodiment of a device for providing nitric oxide for sanitizing or sterilizing an article in a space.

FIG. 4 is a perspective view illustrating another non-limiting embodiment of a device for providing nitric oxide for sanitizing or sterilizing an article in a space.

FIGS. 5A and 5B are photographs illustrating a non-limiting embodiment of the device of FIGS. 1-4.

FIG. 6 is photographs illustrating the effectiveness of a non-limiting embodiment of the device of FIGS. 1-4.

FIGS. 7A-7D are photographs illustrating another non-limiting embodiment of the device of FIGS. 1-4.

FIG. 8 is a photograph illustrating another non-limiting embodiment of the device of FIGS. 1-4.

FIGS. 9A and 9B are photographs illustrating another non-limiting embodiment of the device of FIGS. 1-4.

FIGS. 10A and 10B are photographs illustrating the effectiveness of another non-limiting embodiment of the device of FIGS. 1-4.

FIG. 11 is a chart illustrating the generation of nitric oxide by another non-limiting embodiment of the device of FIGS. 1-4.

FIG. 12 is a chart illustrating the generation of nitric oxide by another non-limiting embodiment of the device of FIGS. 1-4.

FIG. 13 is a chart illustrating the generation of nitric oxide by another non-limiting embodiment of the device of FIGS. 1-4.

FIG. 14 is charts illustrating the generation of nitric oxide by another non-limiting embodiment of the device of FIGS. 1-4.

FIG. 15 is a chart illustrating the generation of nitric oxide by another non-limiting embodiment of the device of FIGS. 1-4.

FIG. 16 is a chart illustrating the generation of nitric oxide by another non-limiting embodiment of the device of FIGS. 1-4.

FIG. 17 is a chart illustrating the generation of nitric oxide by another non-limiting embodiment of the device of FIGS. 1-4.

FIGS. 18A and 18B are perspective views illustrating non-limiting embodiments of a device for providing nitric oxide for sanitizing or sterilizing an article, such as a lumen, in a space.

FIG. 19 is a perspective view illustrating a non-limiting embodiment of an article disposed in a barrier.

FIG. 20 is a perspective view illustrating a non-limiting embodiment of a sleeve containing an article for sterilizing or sanitizing the article.

FIG. 21 is an image illustrating a non-limiting embodiment of a kit for sterilizing or sanitizing an article.

FIG. 22 is a perspective view illustrating a non-limiting embodiment of an article to be sterilized or sanitized.

FIG. 23 is a perspective view illustrating another non-limiting embodiment of a sleeve for the article of FIG. 22.

FIG. 24 is a cross-sectional view illustrating a non-limiting embodiment of device for providing nitric oxide to sanitize or sterilize the article.

FIG. 25 is a cross-sectional view illustrating a non-limiting embodiment of a case for storing the device of FIG. 24.

FIG. 26 is a perspective view illustrating a non-limiting embodiment of another device for providing nitric oxide to sanitize or sterilize the article.

FIG. 27 is another perspective view illustrating a non-limiting embodiment of the device of FIG. 26.

FIG. 28 is another perspective view illustrating a non-limiting embodiment of the device of FIG. 26.

FIG. 29 is another perspective view illustrating a non-limiting embodiment of the device of FIG. 26.

FIG. 30 is another perspective view illustrating a non-limiting embodiment of the device of FIG. 26.

### DETAILED DESCRIPTION

Except in the examples, or where otherwise expressly indicated, all numerical quantities in this description indicating amounts of material or conditions of reaction and/or use are to be understood as modified by the word “about” in describing the broadest scope of the disclosure. In various embodiments, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus the value to which the modifier “about” or “approximately” refers is itself also specifically disclosed.

Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, an “embodiment” means that a particular feature, structure or characteristic is included in at least one or more manifestations, examples, or implementations of this invention. Furthermore, the particular features, structures or characteristics may be combined in any suitable manner, as would be apparent to a person skilled in the art. Combinations of features of different embodiments are all meant to be within the scope of the invention, without the need for explicitly describing every possible permutation by example. Thus, any of the claimed embodiments can be used in any combination.

As used herein, the term “weight percent” (and thus the associated abbreviation “wt. %”) typically refers to a percent by weight expressed in terms of a weight of dry matter. As such, it is to be appreciated that a wt. % can be calculated on a basis of a total weight of a composition, or calculated from a ratio between two or more components/parts of a mixture (e.g. a total weight of dry matter).

As used herein, the term “substantially” refers to the complete, or nearly complete, extent or degree of an action, characteristic, property, state, structure, item, or result. As an arbitrary example, an object that is “substantially” enclosed would mean that the object is either completely enclosed or nearly completely enclosed so as to have the same overall result as if the object were completely enclosed.

The drawings are semi-diagrammatic and not to scale and, particularly, some of the dimensions are for the clarity of presentation and are shown exaggerated in the drawings. Similarly, although the views in the drawings for ease of description generally show similar orientations, this depiction in the drawings is arbitrary. Generally, the device can be operated in any orientation. As used herein, it will be understood that when a first element or layer is referred to as being “over,” “overlying,” “under,” or “underlying” a second element or layer, the first element or layer may be directly on the second element or layer, or intervening elements or layers may be present where a straight line can be drawn through and between features in overlying relationship. When a first element or layer is referred to as being “on” a second element or layer, the first element or layer is directly on and in contact with the second element or layer. Further, spatially relative terms, such as “upper,” “over,” “lower,” “under,” and the like, may be used herein for ease of description to describe one element or feature's relationship to another element(s) or feature(s) as illustrated in the figures. It will be understood that the spatially relative terms are intended to encompass different orientations of the sterilization indicator in use or operation in addition to the orientation depicted in the figures. For example, if the sterilization indicator in the figures is turned over, elements described as being “under” other elements or features would then be oriented “above” the other elements or features. Thus, the exemplary term “under” can encompass either an orientation of above or below. The sterilization indicator may be otherwise oriented (rotated 90 degrees or at other orientations) and the spatially relative descriptors used herein may likewise be interpreted accordingly.

Throughout this disclosure, where publications are referenced, the disclosures of these publications in their entireties are hereby incorporated by reference into this disclosure to more fully describe the state of the art to which this disclosure pertains.

The following detailed description is merely exemplary in nature and is not intended to limit the invention or the application and uses of the invention. Furthermore, there is no intention to be bound by any theory presented in the preceding background or the following detailed description.

### Devices for Providing Nitric Oxide

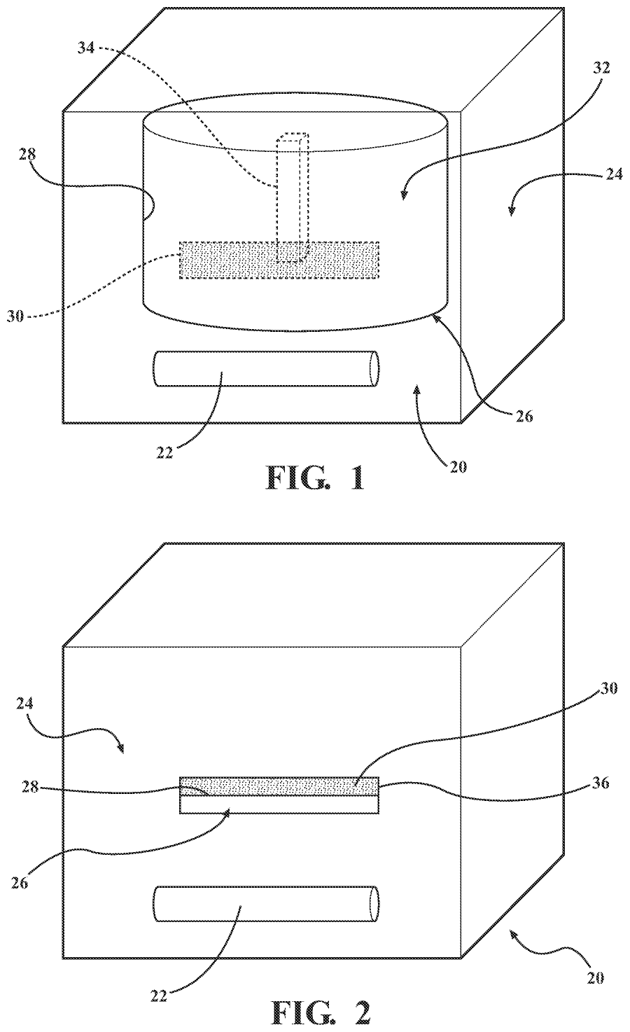

FIGS. 1-4 are perspective views illustrating non-limiting embodiments of devices 20 for providing nitric oxide for sanitizing or sterilizing an article 22 in a space 24. Suitable articles 22 to be sanitized or sterilized include, but are not limited to, personal devices (e.g., mobile phones, tablets, laptops, watches, and the like); personal articles (e.g., face and/or head coverings, sunglasses, credit cards, name badges, keys, jewelry, clothing, accessories, pacifiers, writing tools, and the like); sports equipment (e.g., mouth guards, shoes, and the like); and medical equipment (e.g., instruments, devices, materials, tools, and other equipment that must be sanitary or sterile). It is to be appreciated that any other article can be sanitized or sterilized so long as the article 22 can fit within the space 24 and is compatible with oxides of nitrogen, such as nitric oxide.

Specific examples of suitable medical equipment include, but are not limited to, surgical instruments; cardiac surgery products; cardiac implants; cardiovascular stents; vascular implants; orthopedic surgery products such as surgical instruments, bone graft, bone scaffold; orthopedic implants; dental surgery products; dental implants; endoscopes; gastrointestinal implants, urinary tract implants; wound healing products; tissue engineering products.

The article 22 may be formed from one or more materials, such metals, non-metals, polymeric materials, elastomers, biologically derived materials, and the like. Non-limiting examples of suitable metals include stainless steel, aluminum, nitinol, cobalt chrome, and titanium. Non-limiting examples of non-metals include glass, silica, ceramic, and the like.

The device 20 includes a support 26 adapted to be in proximity of the article 22. In various embodiments, the support 26 may be (A) any object or any combination of objects capable of bearing weight of another object, (B) any object or any combination of objects capable of at least partially impacting movement of a fluid thereby permitting formation of a concentration of a fluid, or both (A) and (B). The term “fluid”, as utilized herein, means any substance that has no fixed shape and yields easily to external pressure, such as a gas or a liquid. Non-limiting examples of suitable supports 26 include containers, such as soft polymeric bags, ethylene oxide pouches, rigid storage containers, and the like; liners, such as fiber-based liners, polymeric liners, metal-containing liners, non-metal liners, and the like; and fibers, such as fiber optic cables and the like.

The support 26 has a surface 28. In some embodiments, the support 26 is a container and has the surface 28 on one or more of its interior surfaces. In other embodiments, the support 26 is a liner and has the surface 28 on one or more of its faces. The surface 28 may have any configuration known in the art (e.g., the surface may or may not be flat). The surface 28 may be smooth, porous, or a combination thereof.

In these and other embodiments, the device 20 may further include a nitric oxide source 30 overlying the surface 28 and adapted to provide nitric oxide. Nitric oxide is very lipid soluble and has the ability to disrupt the lipid membranes of microorganisms. Furthermore nitric oxide may inactivate thioproteins thereby disrupting the functional proteins of microbes. Nitrogen dioxide is more water soluble than nitric oxide. Finally, nitric oxide and nitrogen dioxide are extremely effective disruptors of DNA, causing strand breaks and other damage leading to an inability for the cell to function.

In various embodiments, a mixture of nitric oxide and air will react, resulting in a mixture containing many different oxides of nitrogen. Specifically, the addition of nitric oxide to air, or air to nitric oxide, results in the formation of nitric dioxide when nitric oxide reacts with the oxygen in air. The concentration of each nitrogen-oxide species that is present in a mixture may vary with temperature, pressure, and initial concentration of the nitric oxide.

As used herein, the term “nitric oxide” or “NO” means the NO free radical or NOR. As used herein, the term NOx is an abbreviation for nitrogen oxides or the oxides of nitrogen, which are the oxides formed by nitrogen in which nitrogen exhibits each of its positive oxidation numbers from +1 to +5. As used herein, the terms “nitrogen oxides” and ‘oxides of nitrogen’ and ‘NOx’ mean a gas having one or more of the following gases, all of which contain nitrogen and oxygen in varying amounts: nitric oxide (NO) nitrogen dioxide (NO₂), nitrogen trioxide (NO₃), dinitrogen trioxide (N₂O₃), dinitrogen tetroxide (N₂O₄), dinitrogen pentoxide (N₂O₅) and nitrous oxide (N₂O). As used herein, the phrase “nitric oxide source” means a compound or composition capable of producing or releasing NO, NO₂, and NOₓ.

In certain embodiments, the nitric oxide source 30 includes S-nitroso-N-acetyl-D-penicillamine, nitrite, S-nitrosocysteine, S-nitrosoglutathione, diazeniumdiolate compounds, arginine, organitrites, biological sources adapted to generate nitric oxide, or combinations thereof. The nitric oxide source 30 is adapted to provide nitric oxide (A) at a predetermined rate, (B) for a predetermined amount of time, (C) at a predetermined dose, or any combination of (A), (B), and (C). In one embodiment, the predetermined rate is defined as a rate in an amount of from about 200 parts per billion (ppb) to about 800 ppb and wherein the predetermined amount of time is defined as a time period of from about 2 hours to about 8 hours. In another embodiment, the predetermined rate is defined as a rate in an amount of from about 20 parts per billion (ppb) to about 80 ppb and wherein the predetermined amount of time is defined as a time period of from about 12 hours to about 48 hours.

In yet another embodiment, the nitric oxide source 30 includes a polymer and is adapted to provide nitric acid for a time period of from about 10 to about 200 days, optionally from about 20 to about 150 days, or optionally from about 30 to about 120 days. The polymer may be formed from a silane, a siloxane, or a combination thereof. In certain embodiments, the polymer is polydimethylsiloxane.

In still another embodiment, the predetermined dose is defined as an amount of the nitric oxide at steady state of from about 5 parts per million (ppm) to about 20 ppm.

The article 22 may be sanitized or sterilized after remaining the presence of the nitric acid for at least 5 minutes, optionally at least 10 minutes, or optionally at least 15 minutes. However, it is to be appreciated that sanitization or sterilization times can be impacted by temperature of the space 24, pressure within the space 24, humidity within the space 24, concentration of the nitric oxide within the space 24, presence and concentration of other fluids within the space 24, or combinations thereof.

In exemplary embodiments, the nitric oxide source 30 includes S-nitroso-N-acetyl-D-penicillamine. Non-limiting examples of suitable S-nitroso-N-acetyl-D-penicillamines and other photosensitive S-nitrosothiols covalently attached to polymers are described in U.S. Pat. No. 9,884,943 B2 and International Publication No. WO 2020/018488 A1, which are incorporated by referenced in their entireties.

Referring specifically to FIGS. 1-3, in some embodiments, the support 26 is adapted to be disposed in the space 24. Non-limiting examples of suitable spaces 24 include rooms, such as hospital rooms, classrooms, dining rooms, and the like, and compartments, such as containers, vehicle interiors, glove boxes, and the like.

With particular reference to FIG. 1, in one non-limiting example, the surface 28 of the support 26 defines a cavity 32 with the support 26 adapted to provide fluid communication between the cavity 32 and the space 24. In this example, the device 10 further includes an electromagnetic radiation source 34 adapted to generate electromagnetic radiation within the cavity 32. To this end, the nitric oxide source 30 is disposed within the cavity and adapted to generate nitric acid in the presence of the electromagnetic radiation. In some embodiments, the electromagnetic radiation source 34 is disposed within the cavity 32. However, it is to be appreciated that the electromagnetic radiation source 34 may be located anywhere relative to the support so long as the electromagnetic radiation generated by the electromagnetic radiation source 34 can reach the nitric oxide source 30.

In these and other embodiment, during use of the device 20, the device 20 may be configured as a puck and disposed within the space 24, such as container or a room. The article 22, such as a mobile phone or medical device, may be also disposed within the space 24 and in proximity of the device 20. The electromagnetic radiation source 34, such as LED bulbs, may be activated to generate electromagnetic radiation, such as visible light. The nitric oxide source 30, such as S-nitroso-N-acetyl-D-penicillamine, may provide nitric acid in the presence of the electromagnetic radiation. The article 22 may be sanitized or sterilized after remaining in the presence of the nitric acid for at least 5 minutes, optionally at least 10 minutes, or optionally at least 15 minutes.

With particular reference to FIG. 2, in another non-limiting example, the nitric oxide source 30 includes a polymer and is adapted to provide nitric acid for a time period of from 10 to 200 days. The nitric oxide source 30 including the polymer may be disposed overlying the surface 28 to form a polymer layer 36. The support 26 may include a lignocellulosic material, such as paper.

In these and other embodiment, during use of the device 20, the device 20 may be configured as a card stock coated with the nitric oxide source 30, such as S-nitroso-N-acetyl-D-penicillamine, on its surface 28. The nitric oxide source 30 may include a polymer, such as polydimethylsiloxane, to form the polymer layer 36. The coated card stock may be disposed within the space 24, such as a container or a compartment. The article 22, such as a mobile phone or medical device, may be also disposed within the space 24 and in proximity of the device 20. The polymer layer 36 may provide nitric acid for a time period of from about 10 to about 200 days, optionally from about 20 to about 150 days, or optionally from about 30 to about 120 days. The article 22 may be sanitized or sterilized after remaining in the presence of the nitric acid for at least 5 minutes, optionally at least 10 minutes, or optionally at least 15 minutes.

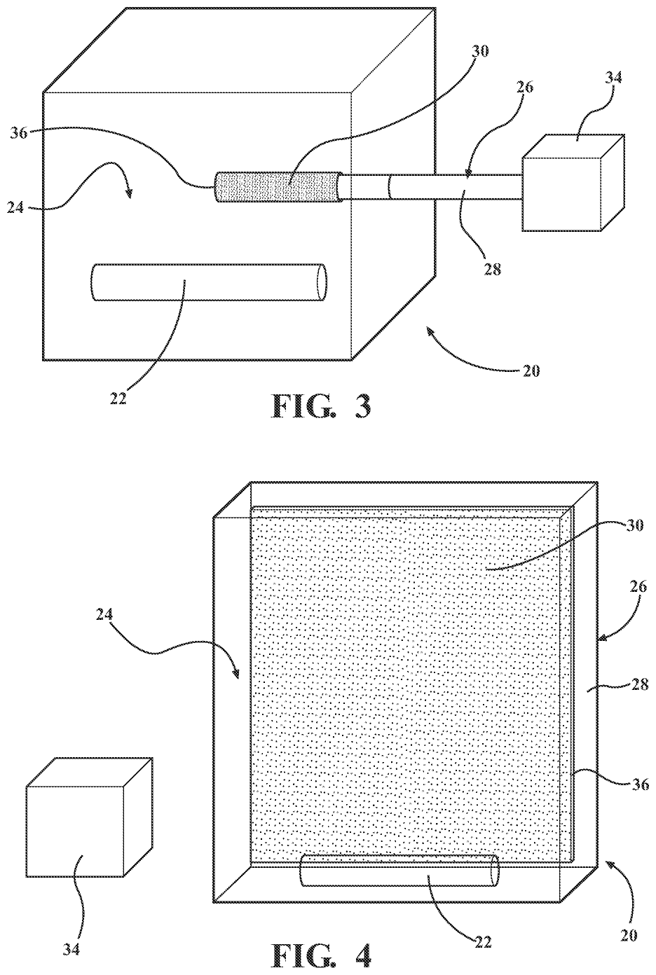

With particular reference to FIG. 3, in yet another non-limiting example, the support 26 includes a filament having the surface 28 with the surface 28 adapted to transmit electromagnetic radiation. In this example, the device 20 further includes an electromagnetic radiation source 34 in optical communication with the filament and adapted to generate electromagnetic radiation. To this end, the nitric oxide source 30 is adapted to provide nitric acid in the presence of the electromagnetic radiation generated by the electromagnetic radiation source 34 and transmitted through the filament.

In these and other embodiment, during use of the device 20, the device 20 may be configured as a fiber optic filament coated with the nitric oxide source 30, such as S-nitroso-N-acetyl-D-penicillamine, on its surface 28. The nitric oxide source 30 may include a polymer, such as polydimethylsiloxane, to form the polymer layer 36. The coated fiber optic filament may be disposed within the space 24, such as a container or a medical device. The article 22, such as a mobile phone or medical device, may be also disposed within the space 24 and in proximity of the device 20. The electromagnetic radiation source 34, such as LED bulbs, may be activated to generate electromagnetic radiation, such as visible light. The visible light may be transmitted through the coated fiber optic cable to the polymer layer 36. The polymer layer 36 may provide nitric acid in the presence of the electromagnetic radiation. The article 22 may be sanitized or sterilized after remaining in the presence of the nitric acid for at least 5 minutes, optionally at least 10 minutes, or optionally at least 15 minutes.

In other embodiments, with reference to FIG. 4, the surface 28 of the support 26 defines the space 24. Non-limiting examples of suitable supports 26 defining spaces 24 include containers, pouches, and the like. The nitric oxide source 30 may overly the surface 28, such as coat the surface 28.

In some embodiments, the nitric oxide source 30 includes a polymer and is adapted to provide nitric acid for a time period of from about 10 to about 200 days, optionally from about 20 to about 150 days, or optionally from about 30 to about 120 days. In other embodiments, the support 26 is substantially transparent to electromagnetic radiation, and the nitric oxide source 30 is adapted to provides nitric acid in the presence of electromagnetic radiation. The device 20 may further include the electromagnetic radiation source 34. However, it is to be appreciated that the electromagnetic radiation source 34 may be located anywhere relative to the support 26 so long as the electromagnetic radiation generated by the electromagnetic radiation source 34 can reach the nitric oxide source 30.

In these and other embodiment, during use of the device 20, the device 20 may be configured as a pouch coated on its interior surface 28 with the nitric oxide source 30, such as S-nitroso-N-acetyl-D-penicillamine. The nitric oxide source 30 may include a polymer, such as polydimethylsiloxane, to form the polymer layer 36. The article 22, such as a mobile phone or medical device, may be disposed within the space 24 (i.e. within the pouch). The polymer layer 36 may provide nitric acid as described immediately above. The article 22 may be sanitized or sterilized after remaining in the presence of the nitric acid for at least 5 minutes, optionally at least 10 minutes, or optionally at least 15 minutes.

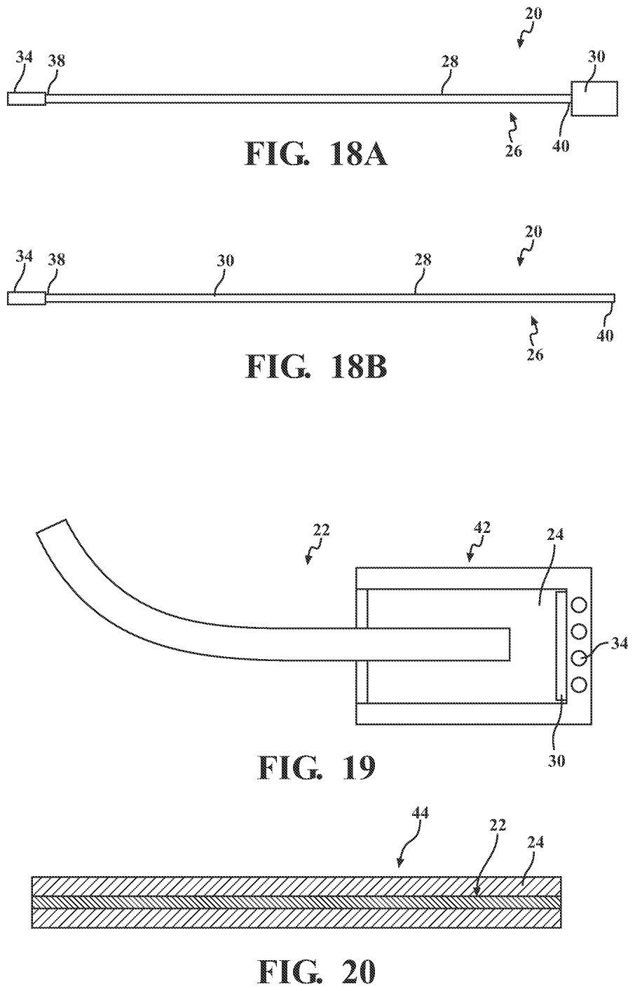

FIGS. 18A and 18B are perspective views illustrating non-limiting embodiments of the device 20 for providing nitric oxide for sanitizing or sterilizing the article 22 defining a void, such as a lumen of an endoscope, in the space 24. In these and other embodiments, the support 26 of the device 20 includes a filament having the surface 28 with the surface 28 adapted to transmit electromagnetic radiation. The filament may have a first end 38 and a second end 40 spaced from the first end 38.

The device 20 may further include the electromagnetic radiation source 34 in optical communication with the filament and adapted to generate electromagnetic radiation. In certain embodiments, the electromagnetic radiation source 34 includes an LED bulb that is coupled to the first end 38.

In one embodiment, the device 20 may further include the nitric oxide source 30 coupled to the second end 40 (see FIG. 18A). In certain embodiments, the nitric oxide source 30 includes SNAP-PDMS or other nitric oxide sources. To this end, the nitric oxide source 30 is adapted to provide nitric acid in the presence of the electromagnetic radiation generated by the electromagnetic radiation source 34 and transmitted through the filament from the first end 38 to the second end 40.

In another embodiment, the device 20 may further include the nitric oxide source 30 disposed on the surface 28 of the support 26 (see FIG. 18B). In certain embodiments, the nitric oxide source 30 includes SNAP-PDMS or other nitric oxide sources. To this end, the nitric oxide source 30 is adapted to provide nitric acid in the presence of the electromagnetic radiation generated by the electromagnetic radiation source 34 and transmitted through the filament from the first end 38 to the second end 40. The device 20 may be disposed within the void of the lumen and, optionally, may be inserted into the lumen all the way to a tip of the endoscope.

FIG. 19 is a perspective view illustrating a non-limiting embodiment of an article 22 disposed in a barrier 42. In some embodiments, the tip of the article 22 or endoscope (i.e. elevator unit), where opening exists, will be covered with a barrier 42 to trap nitric oxide generated by the nitric oxide source 30. The barrier 42 may include an additional nitric oxide source 30 and the electromagnetic radiation source 34. The barrier 42 may define the space 24 proximate to the tip of the endoscope for sterilizing or sanitizing the tip. The nitric oxide can be delivered to the tip of the endoscope from the inside.

FIG. 20 is a perspective view illustrating a non-limiting embodiment of a sleeve 44 containing the article 22 for sterilizing or sanitizing the article 22. In some embodiments, the article 22 may be disposed in the sleeve 44. The sleeve may be coated with the nitric oxide source 30 for emitting nitric oxide for sterilizing or sanitizing an exterior of article 22. The sleeve 44 may be disposable and releases sterilizing agent from the nitric oxide source 30 to maintain sterility for greater than 30 days.

In various embodiments, the surface 28 of the support 26 includes a nitric oxide indicator. Non-limiting examples of suitable indicators are described in U.S. Pat. App. No. 63/141,711, which is incorporated by reference in its entirety. In various embodiments, the indicator is further defined as a dosimeter. The dosimeter may be formed from PDMS, PVC, or the like, and be coated on the surface 28 of the support 28 (e.g., optical fiber, wave guide, and the like). Color change of the dosimeter may be analyzed using UV-VIS spectroscopy.

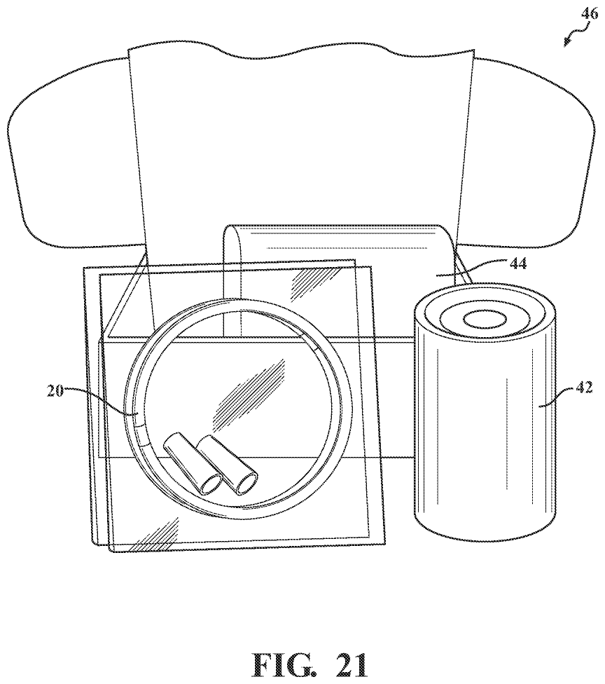

FIG. 21 is an image illustrating a non-limiting embodiment of a kit 46 for sterilizing or sanitizing the article 22. The kit 46 may include the device 20, the barrier 42, and the sleeve 44. In certain embodiments, the kit 46 is adapted for use with an endoscope. The device 20 may be adapted to be disposed within the lumen of the endoscope. The barrier 42 may be adapted to receive the tip of the endoscope. The sleeve 44 may be adapted to receive the endoscope.

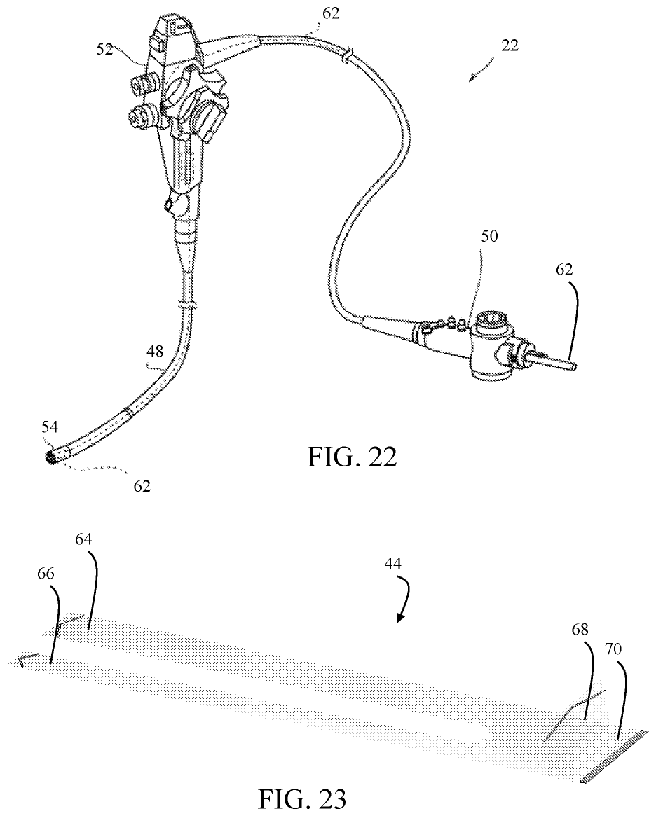

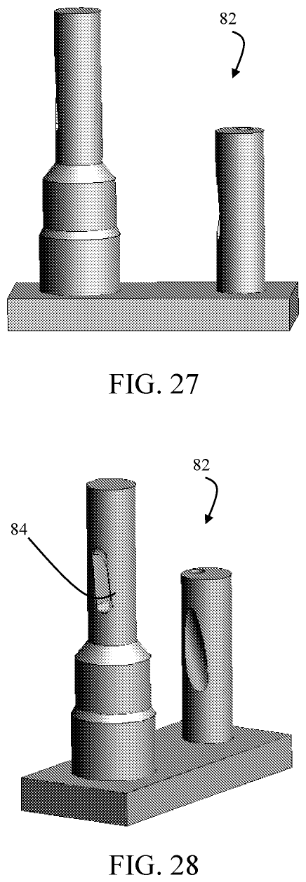

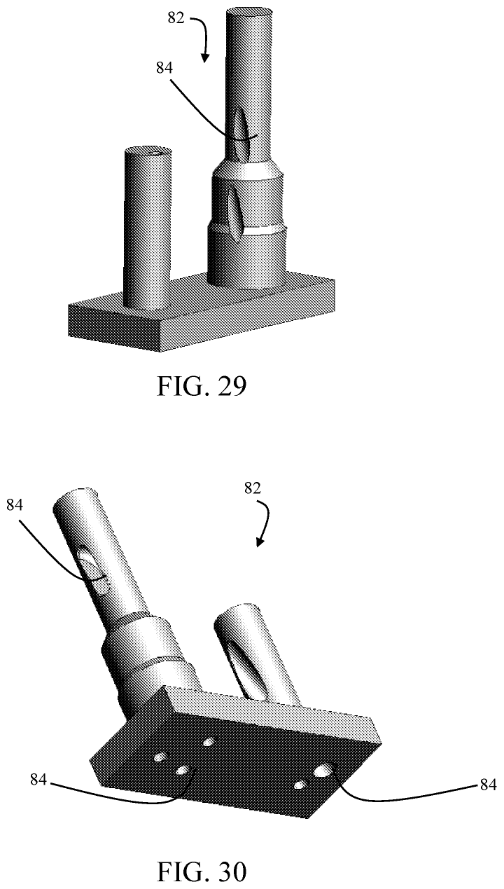

FIGS. 22-30 are various views of components of a system for sterilizing the article 22. With reference to FIG. 22, the system may include the article 22, such as an endoscope. The endoscope may include an insertion section 48, a connection section 50, and a controller section 52 disposed between the insertion section 48 and the connection section 50. The insertion section 48 is adapted for placement in a subject. The insertion section 48 extends to a distal end 54 with the distal end 54 including a light guide hole, a biopsy hole, a lens, and an air/water nozzle. The controller section 52 may include a suction valve 56, an air/water valve 58, and a biopsy channel 60 in fluid communication with the air/water nozzle and the biopsy hole. The connection section 50 includes an optical fiber that extends from the connection section 50 through the controller 52 and to the distal end 54 of the insertion section 48. The support XX may be adapted to be received any of the voids of the article 22 for sterilizing or sanitizing the interior of the article XX.

With reference to FIG. 23, the system may further include the sleeve 44. The sleeve 44 may include a first arm opening 64, a second arm opening 66, and a controller opening 68. The openings 64, 66, 68 may be capable of being sealed and re-opened, for example with a resealable adhesive, when access to the article 22 is desired. The sleeve 44 may further include a slot 70. The slot 70 may be capable of being permanently sealed after the article 22 has been disposed in the sleeve 44. When sterilization of the article 22 is desired, the article 22 may be placed in the sleeve 44 through the slot 70. After the article 22 has been placed in the sleeve 44, the slot 70 may be permanently sealed with an adhesive. Next, the article 22 may be sterilized in the sleeve 44 as described above. When use of the article 22 is desired, one or more of the openings 64, 66, 68 may be opened to access the article 22. It is to be appreciated that the openings 64, 66, 68 may be opened in a certain order depending on the placement of the article 22 therein to prevent contamination of the insertion section 48 while still permitting access to the article 22.

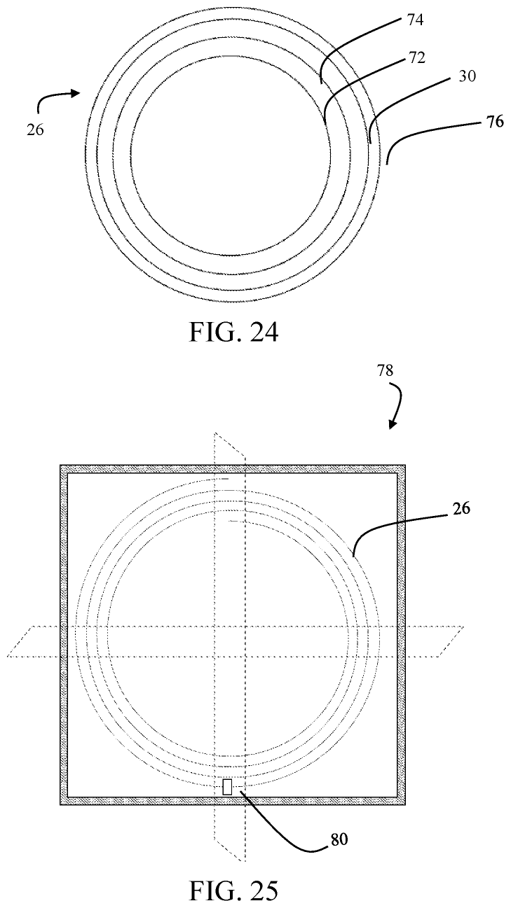

With reference to FIGS. 24 and 25, the support XX may be an optical fiber including a PMMA core 72, a cladding layer 74, the nitric oxide source 30, and a PDMS protective layer 76. The optical fiber may be wound in a case 78 formed from foam to permit insertion of the optical fiber into the article 22 without contaminating the optical fiber. The case 78 may include a cap 80 for accessing the optical fiber.

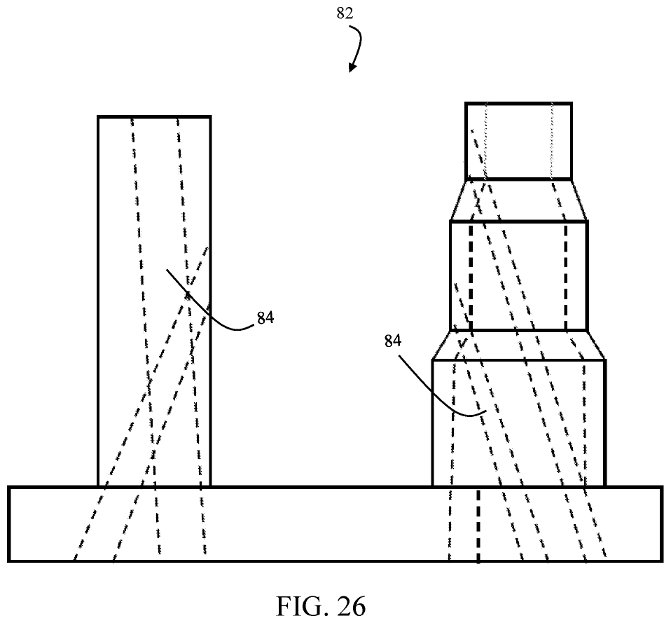

With reference to FIGS. 26-30, the system may further include a guide 82 that is adapted to cooperate with the article 22 to facilitate guidance of the support XX, such as the optical fiber and other components, into the article 22. In particular, the guide 82 is adapted to cooperate with the suction valve 56 and the air/water valve 58 of the endoscope (article 22). Further, the guide 82 defines one or more paths 84 that may be transverse to each other for guiding each of the support XX, such as the optical fibers or other components, through the guide 82 to the suction valve 56 and the air/water valve 58 of the article 22 (e.g., endoscope). The guide 82 includes the nitric oxide source 30 on its surface to sterilize or sanitize areas between the guide 82 and the article 22 that may not be exposed to nitric oxide generated by the sleeve 44 or by other devices.

### Methods for Sanitizing or Sterilizing an Article

Method for sanitizing or sterilizing the article 22 in the space 24 are also provided. The method includes locating the article 22 within the space 24. The space 24 may have a pressure of from about 0.5 atm to about 1.5 atm. The space 24 may have a temperature of from about 10° C. to about 50° C. However, it is to be appreciated that the space 24 may have temperatures and pressures outside of the aforementioned ranges.

The method further includes providing nitric oxide to the space 24 from the nitric oxide source 30 at a predetermined rate. The method further includes exposing the article 22 to the nitric oxide for a predetermined amount of time. In some embodiments, the predetermined rate is defined as a rate in an amount of from about 200 parts per billion (ppb) to about 800 ppb and wherein the predetermined amount of time is defined as a time period of from about 2 hours to about 8 hours. In other embodiments, the predetermined rate is defined as a rate in an amount of from about 20 parts per billion (ppb) to about 80 ppb and wherein the predetermined amount of time is defined as a time period of from about 12 hours to about 48 hours.

The method may further include the step of exposing the nitric oxide source 30 to an initiation condition. The initiation condition may include thermal energy, electromagnetic radiation, pH, water, metal ion mediation, electrochemical initiation, ascorbic acid initiation, vibration, ultrasound, stirring, mechanical agitation, or ultrasonic energy, or combinations thereof. In one exemplary embodiment, the step of exposing the nitric oxide source 30 to the initiation energy is further defined as exposing the nitric oxide source to light.

Although the invention has been described in connection with specific preferred embodiments, it should be understood that the invention as claimed should not be unduly limited to these specific embodiments. While at least one exemplary embodiment has been presented in the foregoing detailed description of the disclosure, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the disclosure as set forth in the appended claims.

### INDUSTRIAL APPLICABILITY

While the present invention is not limited to a particular end application, use or industry, hospitals, schools, restaurants, airlines, and public transit operators often rely on sanitation or sterilization. The devices and methods are useful for using nitric oxide for sanitation or sterilization.

The following examples, illustrating the devices and methods of this disclosure, are intended to illustrate and not to limit the invention.

### EXAMPLES

The following examples are included to demonstrate various embodiments as contemplated herein. It should be appreciated by those of skill in the art that the techniques disclosed in the examples which follow represent techniques discovered by the inventor(s) to function well in the practice of the invention, and thus can be considered to constitute desirable modes for its practice. However, those of skill in the art should, in light of the present disclosure, appreciate that many changes can be made in the specific embodiments which are disclosed and still obtain a like or similar result without departing from the spirit and scope of the invention. All percentages are in wt. % and all measurements are conducted at 23° C. unless indicated otherwise.

### Example 1: Antimicrobial Effectiveness of No Gas Sterilization System

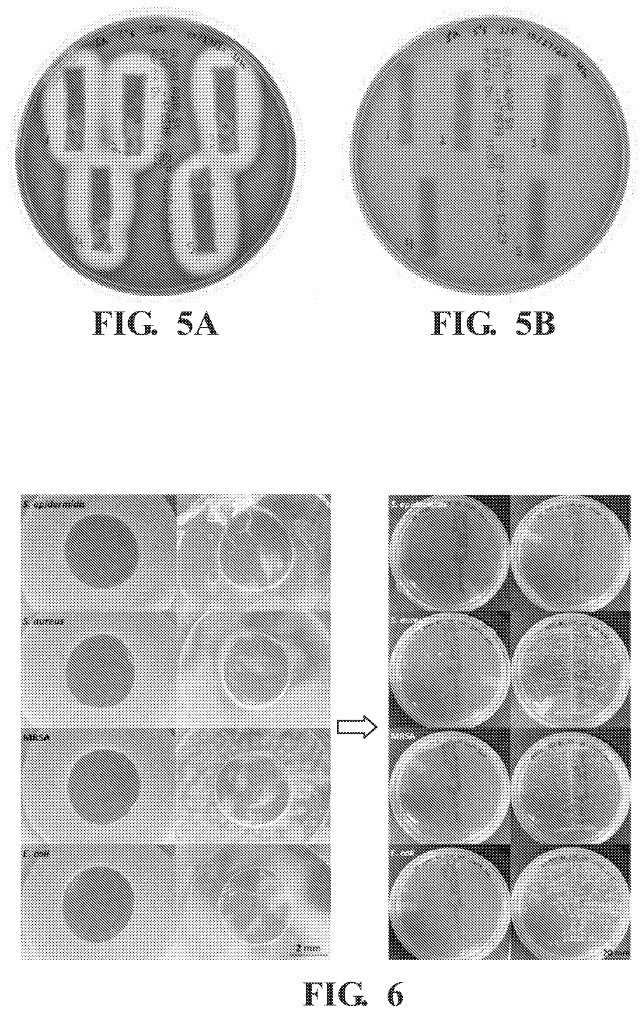

Strips of filter paper were inoculating with S. aureus and then threated for 4 hours with a control that did not release nitric oxide (“NO”) or gas phase NO from SNAP-PDSM illuminated with light. The filter paper was then placed on blood agar plates and incubated for 48 hours to allow any viable bacteria ample time to proliferate. As shown in FIG. 5A, the yellow color that develops is the result of viable bacteria growing. As shown in FIG. 5B, the absence of yellow color indicates no viable bacteria is present. FIG. 5 shows photographs of the blood agar plates after NO treatment and incubation. FIG. 6 shows antimicrobial effects of NO generated from SNAP-PDMS against S. Epidermis, S. aureus, MRSA, and E. coli. When discs of the polymer were placed on streaked plates. The centers of the discs were removed and used to inoculate new streak plates to prove there was no viable bacteria present after treatment.

### Example 2: No Generating Puck

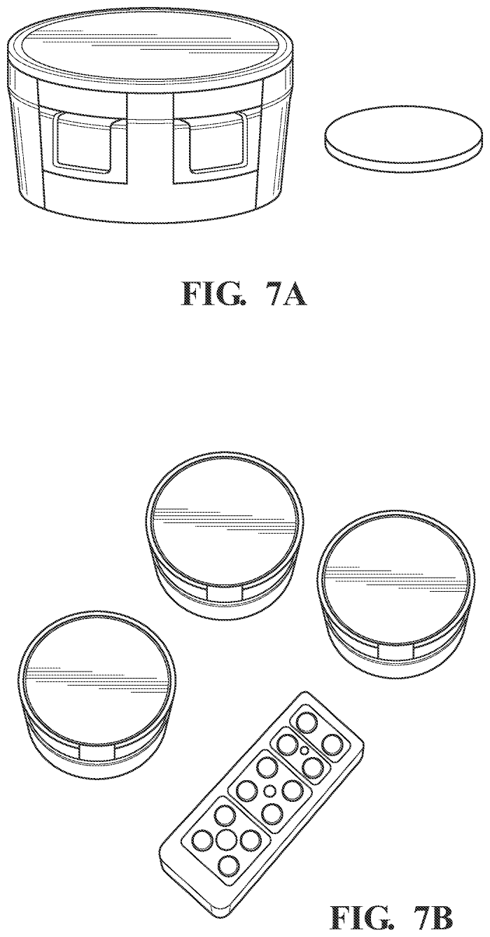

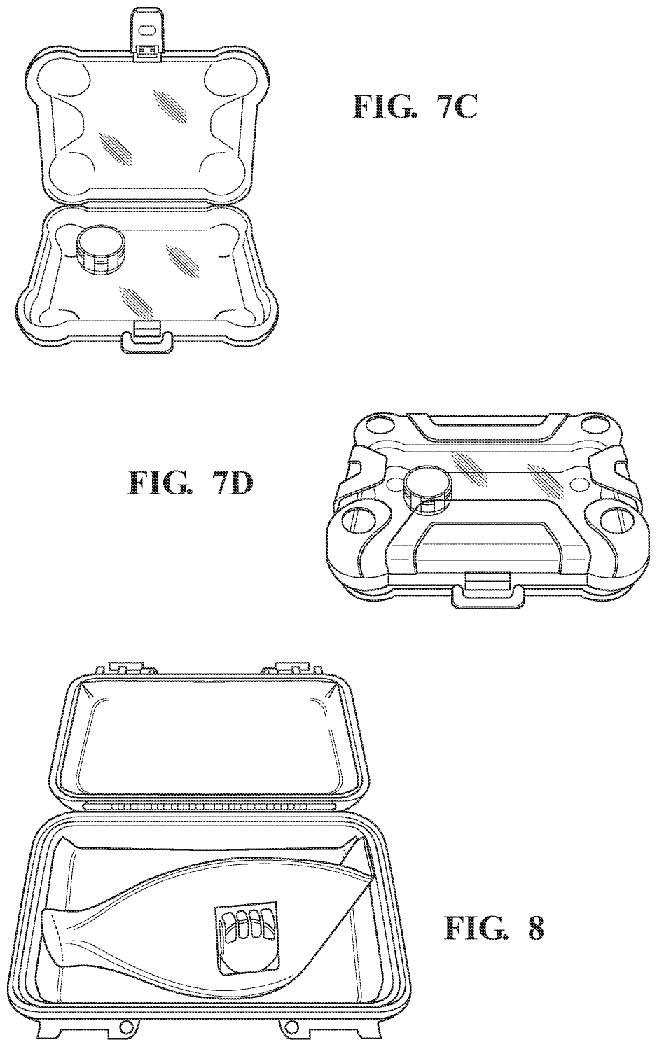

A plastic box for personal electronics with an NO generating puck that uses photolytic generation of NO is shown in FIG. 7. A small LED light source is used to illuminate a polymer ring of SNAP-PDMS. When light impinges on this polymer ring, NO is generated to a level that reaches a steady state of approximately 12 ppm. This device is places in the box with items that the user desires to sterilize (for regulated use) or sanitize (for personal use), turns the light on the puck on and closes the box. NO accumulates in the box and microorganisms/infectious agents are inactivated and destroyed. The total amount of NO generated by the puck is substantially less than the OSHA limit of NO exposure (we have more information regarding this, but we are almost 2 orders of magnitude lower than the OSHA limit). The following can be tuned: size and shape of the box, the total amount of NO in the puck, the intensity of light in the puck, and the number of pucks used at the same time. There are also other details such as switch placement, battery life, remote control to turn on/off that will be specified depending on what is being sterilized or sanitized.

Panel A shows the NO generating puck that uses light to generate NO. When the light is turned on, NO is actively generated. Panel B shows a second type of NO generating puck that uses a remote-control switch with a timer and dimmer switch that allows external control of the NO generation but controlling the light generated. Panels C and D are photographs of a plastic box that the puck is placed to create a contained box that is able to sanitize items in the box once the light is turned on.

### Example 3: No Generating Coated Paper in a Container

Plastic box for sanitizing face masks using NO releasing polymer coated paper is shown in FIG. 8. The coated paper (card stock) is coated with PVC that contains blended SNAP. NO is generated continuously from the paper for 30-120 days, depending on the specific polymer formulation used. The level of NO that accumulates in the box effectively sanitizes face masks placed in the box for at least 15 minutes. The NO is not actively triggered to be released, but rather is passively released continuously. The paper liner is replaced after the specified time limit.

### Example 4: No Generating Coated Paper in a Compartment

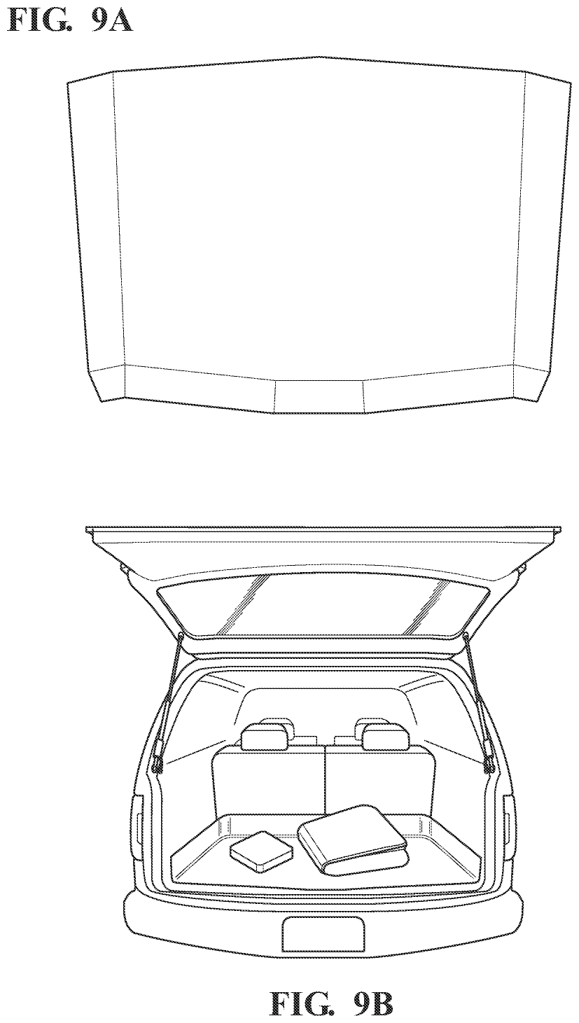

A vehicle electronics compartment/glove box is shown in FIG. 9. The coated paper from Example 3 is disposed in the electronic compartments/glove box of vehicles, creating a chamber in a car that will sanitize masks, keys, credit cards, etc. in a person's vehicle immediately after use. The paper liner is replaced periodically to maintain the ability of the compartment to sanitize items placed in the compartment.

Panel A shows the coated paper insert that is cutout, folded and place in the compartment. Panel B shows the insert in place with personal items to be sanitized. The paper passively and continuously releases NO for 30-120 days, depending on the specific polymer formulation used to manufacture the paper liners.

### Example 5: No Generating Sterilization Pouch

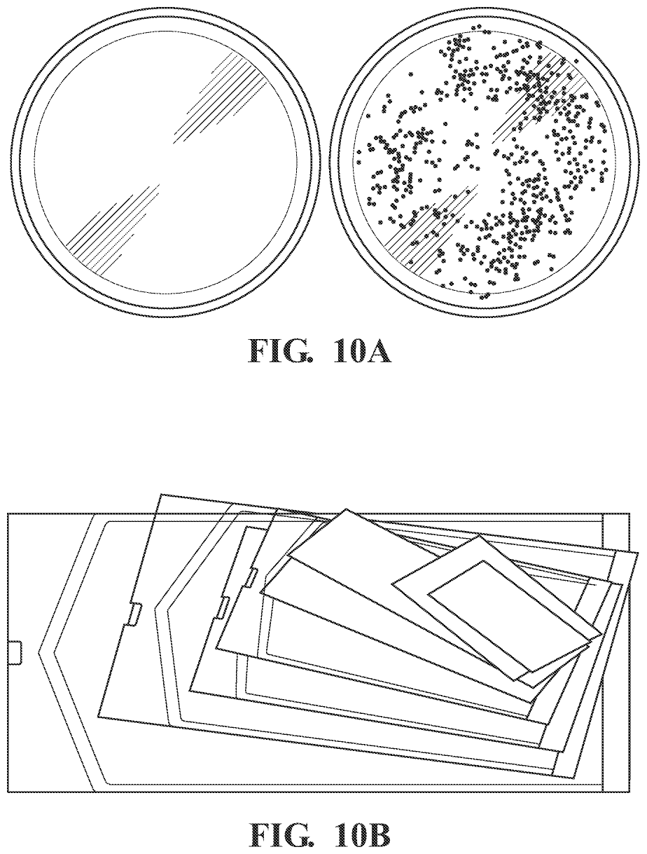

A sterilization pouch is shown in FIG. 10. Soft, disposable sterilization pouches are fabricated to sterilize to FDA standards medical devices placed within the pouch. The pouch (could be made of many materials such as foil, Tyvek, sealed paper, etc.) contains a strip, a coating, or a piece of SNAP-PDMS on the inner surface so that when it is seal, the interior of the pouch will generate enough NO to sterilize the items placed within it. FIG. 10 provides results for sterilization using the pouch that includes SNAP-PDMS as compared to a control pouch that did not include an NO source. Both pouches were inoculated with the same load of bacteria at the same time. The NO can be triggered to be released by light or passive thermal decomposition. The items will remain sterile until the integrity of the pouch is compromised. This allows passive sterilization at room temp and pressure and allows long term storage until the device is needed.

Panel A shows photographs of the results of 3 hours of bacterial challenge to a sealed foil pack to demonstrate effectiveness in sterilizing the space within a sealed package. This specific image was taken of S. epidermidis loaded in the pouch that contained SNAP-PDMS (left) and control (right) after 3 hours incubation at 37° C. Panel B shows examples of a variety of sterilization pouches that could be used to create “containers” for sterilizing medical devices.

### Example 6: No Generating Filament

Fiber optic filament coated with SNAP-PDMS was formed. Various levels of NO were generated depending on coating thickness on the declad fiber optic element with a light source coupled to the fiber and illuminating the optical fiber such that light is propagated down the fiber and interacts with the SNAP-PDMS. This specific example provides a PMMA fiber coated with different thickness of SNAP-PDMS when illuminated with a 470 nm LED. This coated filament can be placed in the lumen of an endoscope where the lumen itself serves as the confining container. Upon illuminated, the appropriate level of NO is generated such that the lumen of the endoscope is sterilized.

### Example 7: Evaluation of Various No Generating Devices

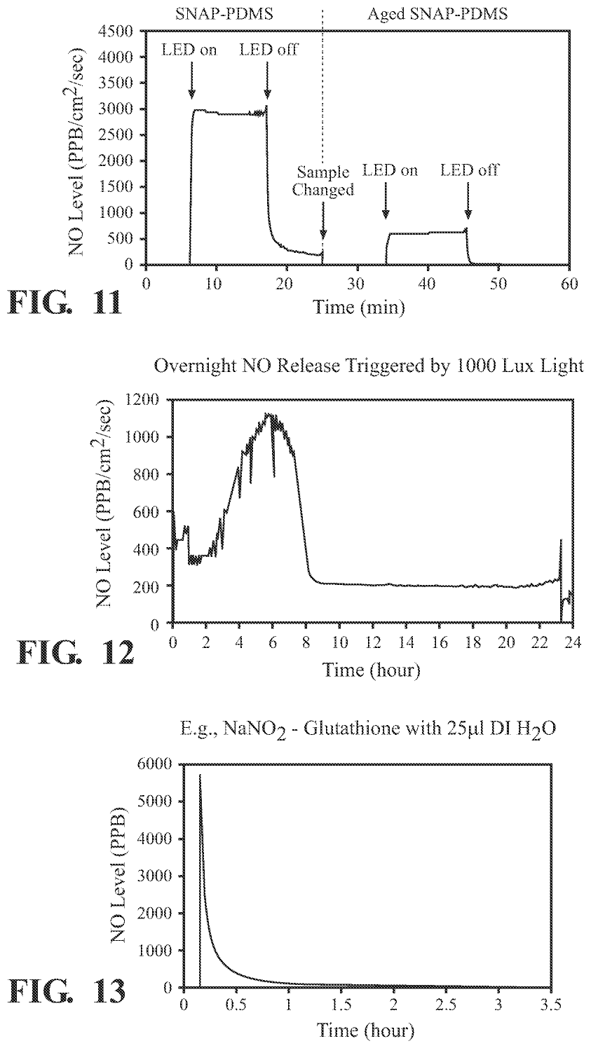

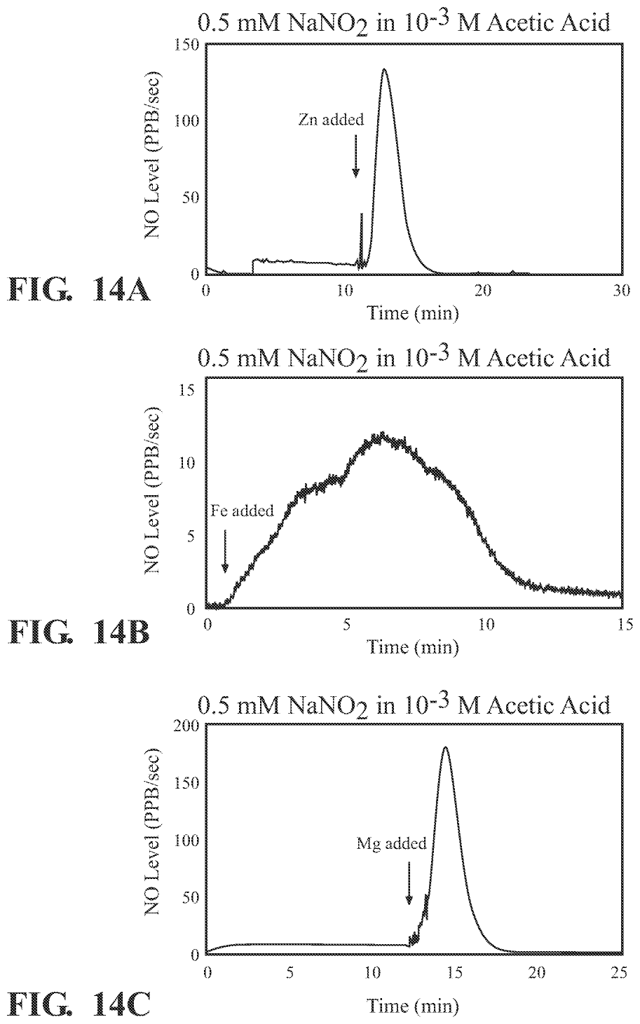

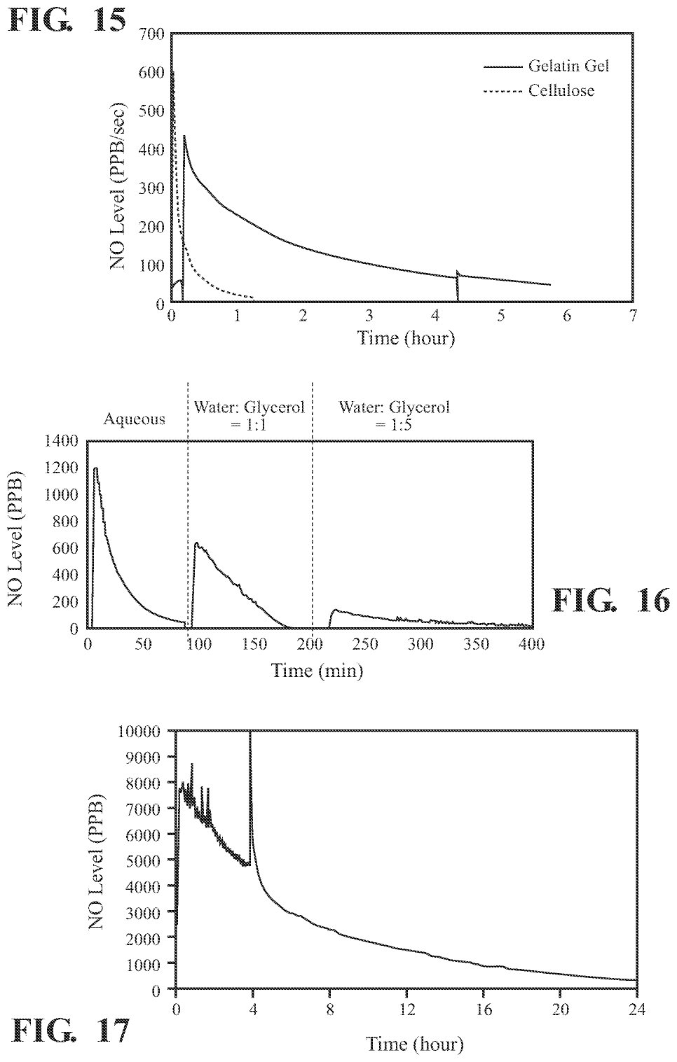

Various NO generating devices were formed and evaluated for NO release. FIG. 11 provides an evaluation of NO release from SNAP-PDMS film with a LED light trigger. FIG. 12 provides an evaluation of NO release from SNAP-PDMS film with an incandescent light trigger. FIG. 13 provides an evaluation of NO release from sodium nitrite in water with glutathione added. FIG. 14 provides an evaluation of NO release from sodium nitrite in a mild acid with the addition of a metal ion (Zn, Fe, Mg) being the trigger. FIG. 15 provides an evaluation of NO release from sodium nitrite in different hydrogels (gelatin gel or cellulose) with each combined with ascorbic acid. FIG. 16 provides an evaluation of NO release from sodium nitrite in water and glycerol in different ratios to modulate the NO release profile using ascorbic acid as the trigger. FIG. 17 provides an evaluation of NO release from sodium nitrite and ascorbic acid embedded in foam dressings with added water to initiate release.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A device for providing nitric oxide to a space for sanitizing or sterilizing an article, the device comprising:
a support having a surface, the support adapted to be disposed in the space and in proximity of the article; and
a nitric oxide source overlying the surface and adapted to provide nitric oxide:
(A) at a predetermined rate;
(B) for a predetermined amount of time;
(C) at a predetermined dose; or
any combination of (A), (B), and (C).

2. The device of claim 1, wherein:
the surface of the support defines a cavity with the support adapted to provide fluid communication between the cavity and the space;
the device further comprises an electromagnetic radiation source adapted to generate electromagnetic radiation within the cavity; and
the nitric oxide source is disposed within the cavity and adapted to provide nitric acid in the presence of the electromagnetic radiation.

3. The device of claim 2, wherein the electromagnetic radiation source is disposed within the cavity.

4. The device of claim 1, wherein the nitric oxide source comprises a polymer and is adapted to provide nitric acid for a time period of from about 10 to about 200 days.

5. The device of claim 4, wherein:
(A) the nitric oxide source is disposed overlying the surface to form a polymer layer;
(B) the support comprises a lignocellulosic material; or
both (A) and (B).

6. The device of claim 1, further wherein:
the support comprises a filament, the filament has the surface, and the surface is adapted to transmit electromagnetic radiation;
the device further comprises an electromagnetic radiation source in optical communication with the filament and adapted to generate electromagnetic radiation; and
the nitric oxide source is adapted to provide nitric acid in the presence of the electromagnetic radiation.

7. A device for providing nitric oxide to a space for sanitizing or sterilizing an article, the device comprising:
a support having a surface, the surface defining the space, the space adapted to contain the article; and
a nitric oxide source overlying the surface and adapted to provide nitric oxide:
(A) at a predetermined rate;
(B) for a predetermined amount of time; or
(C) at a predetermined dose;
any combination of (A), (B), and (C).

8. The device of claim 7, wherein the nitric oxide source comprises a polymer and is adapted to provide nitric acid for a time period of from about 10 to about 200 days.

9. The device of claim 7, wherein the support is substantially transparent to electromagnetic radiation, and the nitric oxide source is adapted to provide nitric acid in the presence of electromagnetic radiation.

10. The device of claim 7 further comprising an electromagnetic radiation source adapted to generate electromagnetic radiation within the space, wherein the nitric oxide source is adapted to generate nitric acid in the presence of the electromagnetic radiation.

11. A method for sanitizing or sterilizing an article in a space, the method comprising:
locating the article within the space;
providing nitric oxide to the space from a nitric oxide source at a predetermined rate; and
exposing the article to the nitric oxide for a predetermined amount of time.

12. The method of claim 11, wherein:
the predetermined rate is defined as a rate in an amount of from about 200 parts per billion (ppb) to about 800 ppb and wherein the predetermined amount of time is defined as a time period of from about 2 hours to about 8 hours; or
the predetermined rate is defined as a rate in an amount of from about 20 parts per billion (ppb) to about 80 ppb and wherein the predetermined amount of time is defined as a time period of from about 12 hours to about 48 hours.

13. The method of claim 11, wherein the predetermined dose is defined as an amount of the nitric oxide at steady state of from about 5 parts per million (ppm) to about 20 ppm.

14. The method of claim 11, wherein the nitric oxide source comprises S-nitroso-N-acetyl-D-penicillamine, nitrite, S-nitrosocysteine, S-nitrosoglutathione, diazeniumdiolate compounds, arginine, organitrites, biological sources adapted to generate nitric oxide, or combinations thereof.

15. The method of claim 11, wherein the nitric oxide source further comprises a polymer.

16. The method of claim 15, wherein the polymer is formed from a silane, a siloxane, or a combination thereof.

17. The method of claim 11, further comprising the step of exposing the nitric oxide source to an initiation condition comprising thermal energy, electromagnetic radiation, pH, water, metal ion mediation, electrochemical initiation, ascorbic acid initiation, vibration, ultrasound, stirring, mechanical agitation, or ultrasonic energy, or combinations thereof.

18. The method of claim 17, wherein the step of exposing the nitric oxide source to an initiation energy is further defined as exposing the nitric oxide source to light.

19. The method of claim 11, wherein the space has a pressure of from about 0.5 atm to about 1.5 atm.

20. The method of claim 11, wherein the space has a temperature of from about 10° C. to about 50° C.
